(19) United States
(12) Patent Application Publication
(10) Pub. No.: US 2025/0303004 A1
(43) Pub. Date: Oct. 2, 2025

(54) RADIOPHARMACEUTICAL COMPOSITIONS FOR LOW TOXICITY ACTINIUM IN TARGETED RADIONUCLIDE THERAPY

(71) Applicant: Curium US LLC, St. Louis, MO (US)

(72) Inventors: Jarno Jalomäki, Helsinki (FI); Salla Seppänen, Helsinki (FI)

(73) Assignee: Curium US LLC, St. Louis, MO (US)

(21) Appl. No.: 18/681,264

(22) PCT Filed: Aug. 3, 2022
(86) PCT No.: PCT/US2022/074477

§ 371 Date: Feb. 5, 2024

Related U.S. Application Data
(60) Provisional application No. 63/229,061, filed on Aug. 3, 2021.

(51) Int. Cl. A61K 51/04; A61K 47/10; A61K 47/22; A61P 35/00

(52) U.S. Cl. CPC A61K 51/0497; A61K 47/10; A61K 47/22; A61P 35/00

(57) ABSTRACT

The present disclosure provides a high-energy, low toxicity radiopharmaceutical composition comprising actinium that performs as an anti-tumor agent for targeted radionuclide therapy. The radiopharmaceutical composition disclosed herein has a short physical half-life of about 9.9 days and produces low toxicity profiles.

### RELATED APPLICATION

This application claims the benefit of U.S. Patent Application No. 63/229,061, filed Aug. 3, 2021, which is incorporated herein by reference in its entirety to the full extent permitted by law.

### FIELD OF THE INVENTION

The present disclosure relates to a high-energy, low toxicity radiopharmaceutical composition comprising actinium that performs as an anti-tumor agent for targeted radionuclide therapy.

### BACKGROUND OF THE INVENTION

Prostate cancer (PC) is the most frequent non-cutaneous cancer and the second most frequent cause of cancer deaths for adult men. A worldwide estimate of PC in 2008 implied 899,000 new cases and 258,000 PC deaths. Most deaths related to prostate cancer are due to advanced disease, which results from any combination of lymphatic, blood, or contiguous local spread. Most patients with PC who die, die of metastatic PC.

Targeted radionuclide therapy has become an attractive and quickly developing therapy option for many different cancers, such as lymphoma, melanoma, and neuroendocrine tumors.

During the last decade, six new drugs have been found to increase overall survival for patients with metastatic castration-resistant prostate cancer (mCRPC). Patients with symptomatic mCRPC have initially been treated with docetaxel. Abiraterone, enzalutamide, cabazitaxel, sipuleucel, and radium-223 increase overall survival for patients who had failed treatment with docetaxel. However, randomized trials have not evaluated the drugs for patients with failure in response to second-line treatment following recurrence after docetaxel. Therefore, international organizations such as European Association of Urology (EAU)/European Society of Radiotherapy and Oncology (ESTRO) have guidelines but no recommendations for third-line treatment of mCRPC.

Due to unmet needs, the St. Gallen Advanced Prostate Cancer Consensus Conference (APCCC) 2017 favored third-line treatment with cabazitaxel and with androgen receptor (AR) and AR signaling inhibitors. Of PC, poorly differentiated, metastatic, and hormone refractory adenocarcinomas of the prostate express prostate specific membrane antigen (PSMA) and 68Ga-PSMA HBED-CC PET/CT detects sites of cancer lesions for most patients with mCRPC. Patients with a positive 68Ga-PSMA HBED-CC PET/CT might be treated with PSMA radioligand therapy (RLT).

In PC, after unsuccessful therapy with 90Y-CYT-356 monoclonal antibody (mAb), which binds to the intracellular domain of PSMA, Phase 1 and 2 clinical trials utilizing the PSMA mAb J591, radiolabeled with ¹⁷⁷Lu or ⁹⁰Y, showed promising results; however, there were higher rates of haematological toxicity. In 47 patients treated with ¹⁷⁷Lu-PSMA mAb J591 grade 4 thrombocytopenia in 46.8% (29.8% received platelet transfusions) was reported and a total of 25.5% experienced grade 4 neutropenia. Monoclonal antibodies are large molecules and therefore show poor permeability in solid tumors. This characteristic along with slow clearance from the circulation is the probable cause of grade 4 haematotoxicity.

Due to the side effects, there is a significant disadvantage in using Lu-J591. It is therefore prudent to consider small molecule inhibitors of PSMA instead of mAb. ¹⁷⁷Lu-PSMA-617 and ¹⁷⁷Lu-PSMA I&T are small-molecule inhibitors of PSMA that are extremely desirable for targeted radionuclide therapy due to their low haematotoxicity and nephrotoxicity profiles, providing better effects and fewer adverse effects than ¹⁷⁷Lu-J591.

Despite this, there are cases where patients fail to be affected by ¹⁷⁷Lu-PRLT treatment. In these instances, clinical application of ²²⁵Ac-PSMA targeted alpha therapy (TAT) as last line of therapy in patients with mCRPC has demonstrated an excellent response, e.g., chemotherapy naive patients, although most clinical studies report it as third-line therapy or after a failure of ¹⁷⁷Lu-PRLT.

Widespread application of ²²⁵Ac-PSMA TAT is hampered by its salivary gland toxicity. Therefore, there exists a clinical need for an effective treatment for mCRPC with lower rates of toxicity. ²²⁵Ac-PSMA-617 is a new and promising therapy option for patients with mCRPC which contains the advantages of previous methods of treatments with low rates of toxicity.

### SUMMARY OF THE INVENTION

Among the various aspects of the present disclosure is a radiopharmaceutical composition comprising actinium that performs as an anti-tumor agent for targeted radionuclide therapy. The composition, when administered to a subject, results in low toxicity profiles, providing better effects and fewer adverse effects than monoclonal antibody treatments and other comparable third-line treatments.

Another aspect of the present disclosure is a pharmaceutical composition comprising an ²²⁵Ac-PSMA-I&T solution for injection containing actinium, ascorbic acid, and ethanol; wherein the ²²⁵Ac-PSMA-I&T is in sufficient amounts of radioactivity for intended use; wherein the concentration of ascorbic acid is about 35 to 45 mg and the total amount of ethanol is about 50 to 80 mg; wherein upon administration of the composition to a subject, the subject maintains low levels of toxicity; and wherein the prostate-specific antigen decline is more than about 50%.

Other features and aspects of the disclosure are described in detail below.

### Reference to Color Figures

This application file contains at least one figure executed in color. Copies of this patent application publication with color drawings will be provided by the Office upon request and payment of the necessary fee.

### BRIEF DESCRIPTION OF THE DRAWINGS

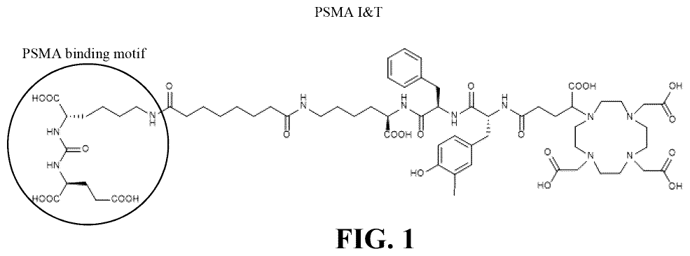

FIG. 1 presents the structural formula of the precursor PSMA I&T.

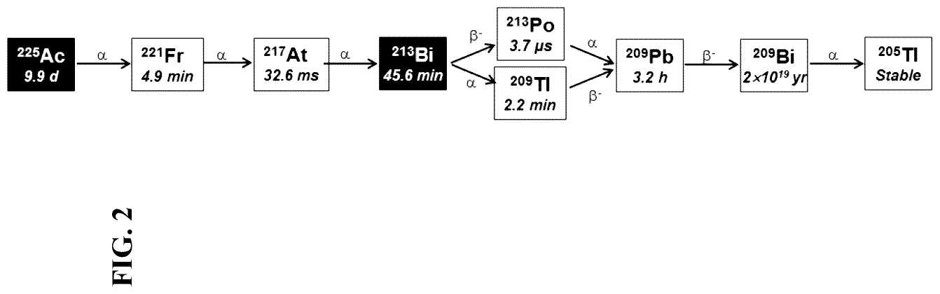

FIG. 2 shows the Ac-225 decay scheme.

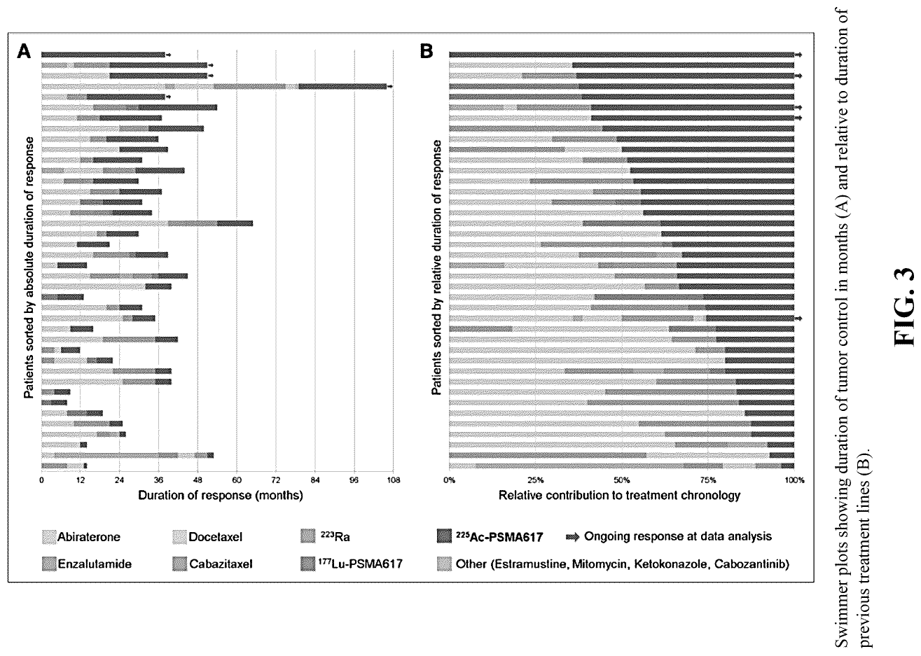

FIG. 3 depicts therapy sequences, lengths in an Ac-225-PSMA trial.

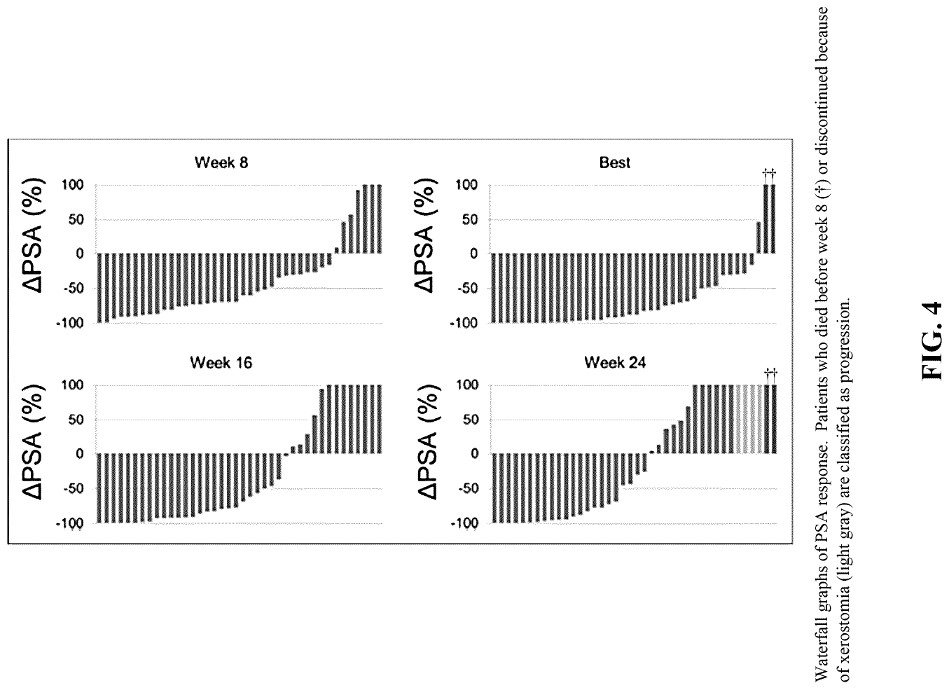

FIG. 4 depicts PSA responses in an Ac-225-PSMA trial.

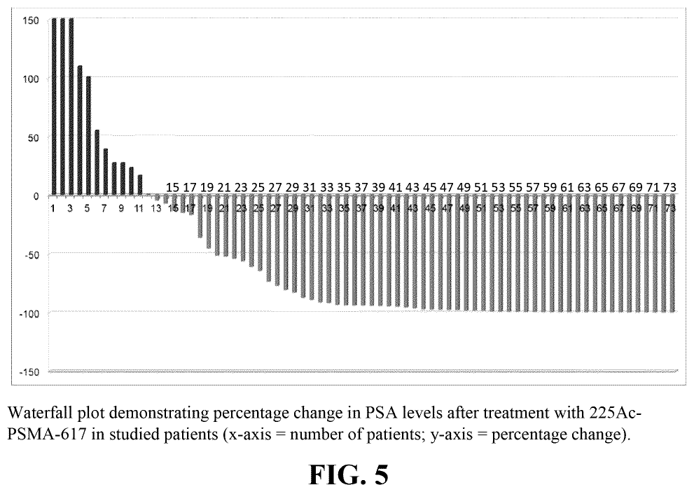

FIG. 5 depicts PSA responses in 73 pts in the largest Ac-225-PSMA trial.

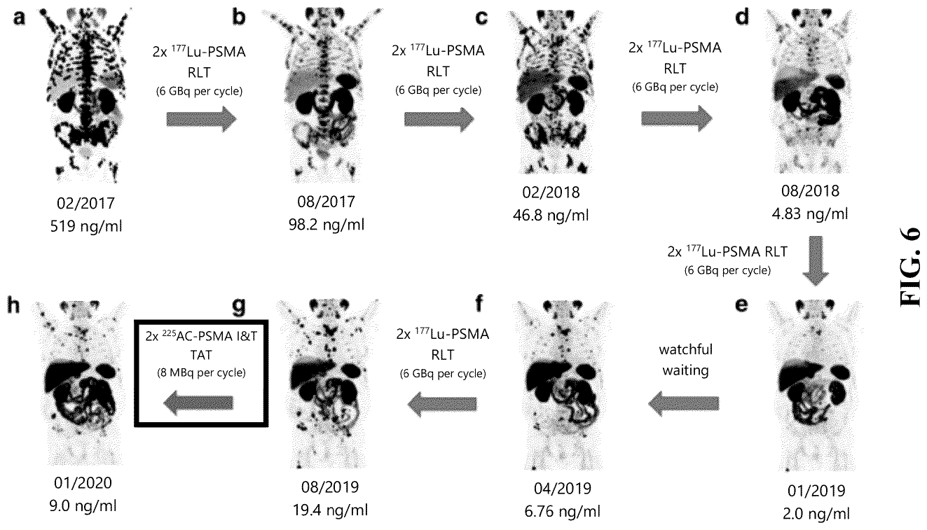

FIG. 6 depicts a response with Ac-225-PSMA after 10 cycles of Lu-177-PSMA.

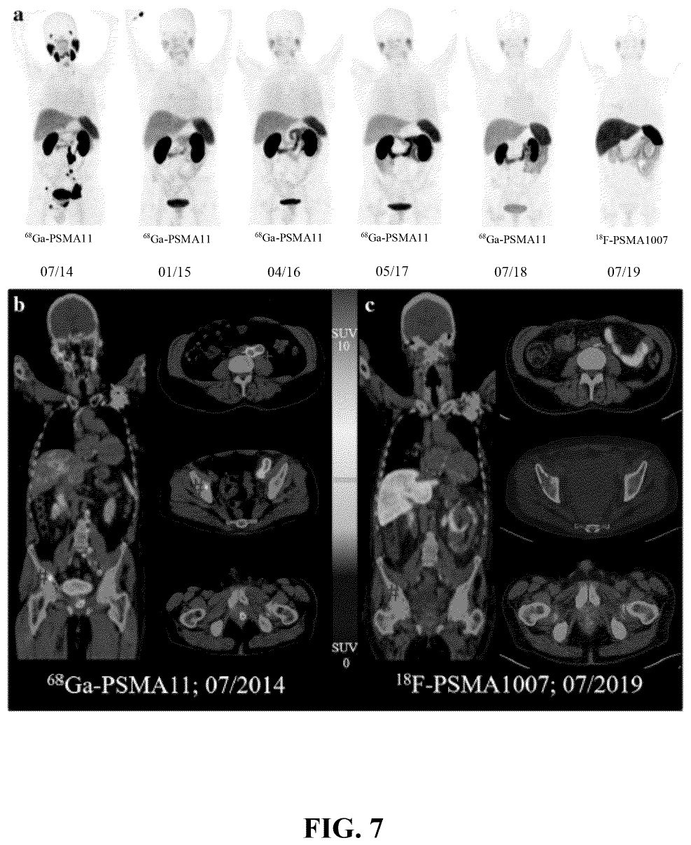

FIG. 7 depicts a 5-year complete response with Ac-225.

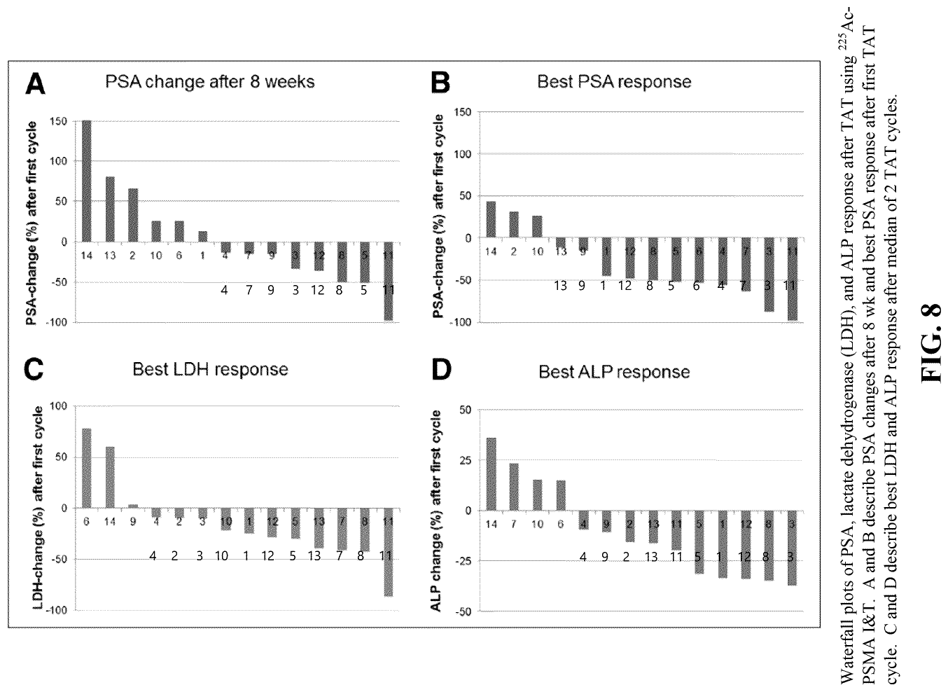

FIG. 8 depicts PSA-response with Ac-225-PSMA-I&T.

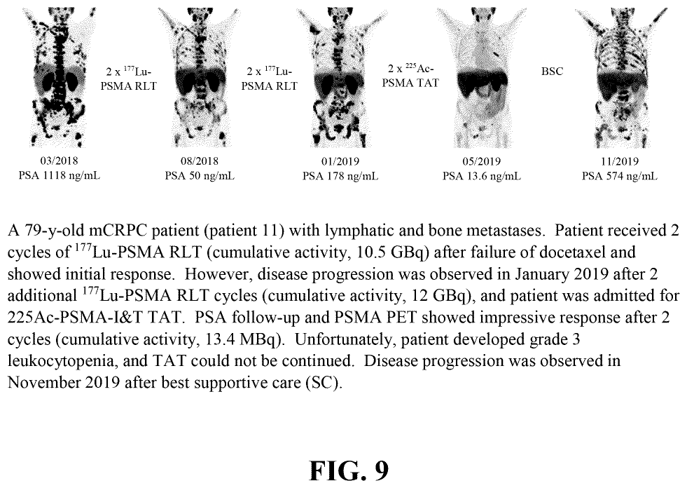

FIG. 9 depicts a patient treated with Ac-225-PSMA-I&T.

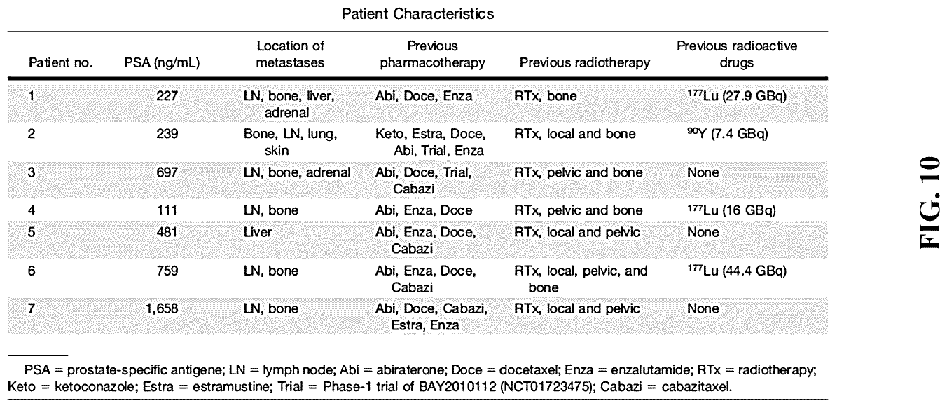

FIG. 10 depicts patient characteristics with poor response to Ac-225-PSMA-617.

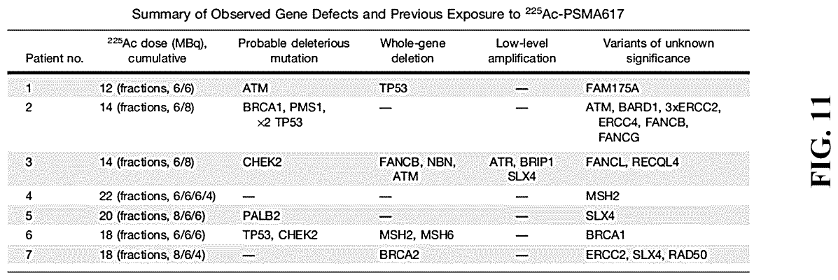

FIG. 11 depicts gene defects by NGS in patients with poor response to Ac-225-PSMA-617

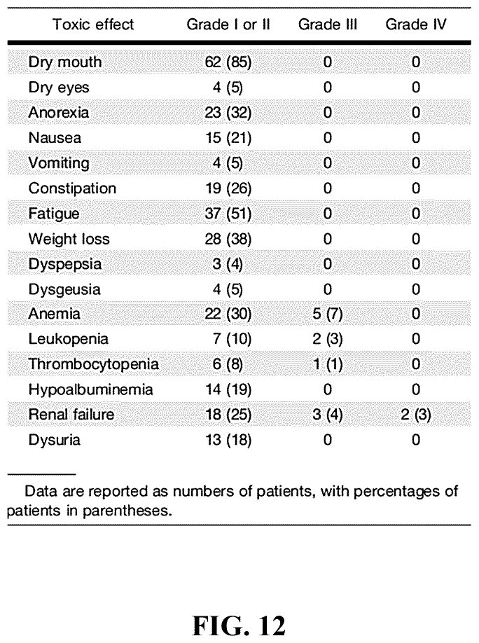

FIG. 12 depicts toxicities in 73 patients after Ac-225-PSMA.

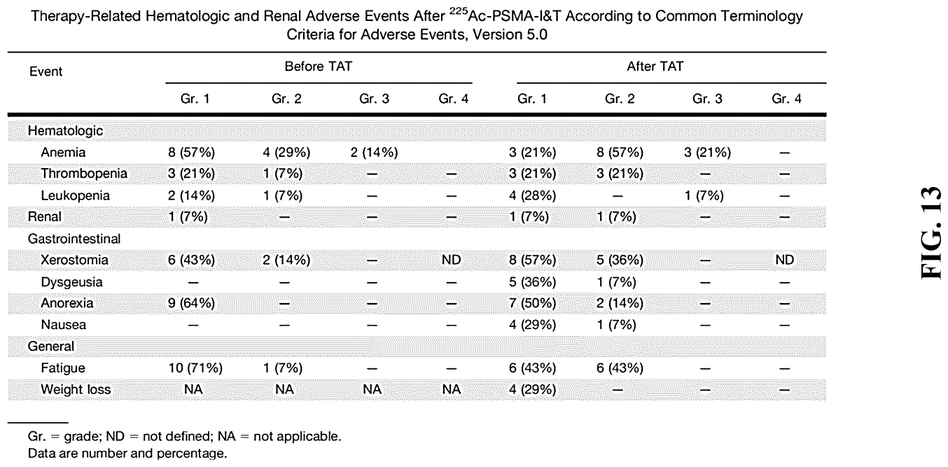

FIG. 13 depicts hematologic and kidney adverse events after Ac-225-PSMA-I&T.

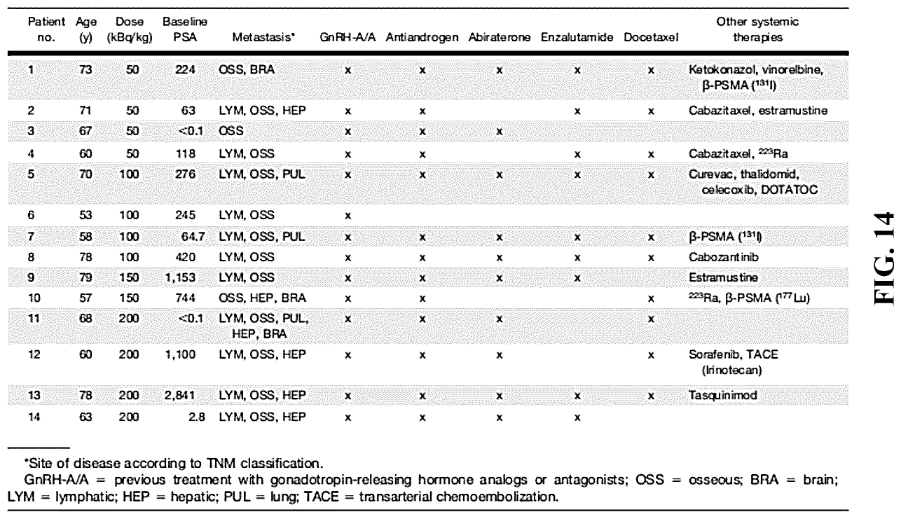

FIG. 14 depicts patient characteristics of the dose finding study of Ac-225-PSMA-617.

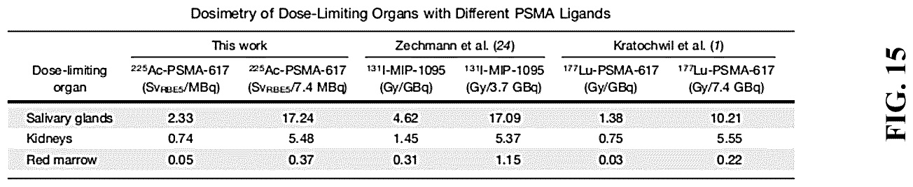

FIG. 15 depicts the dosimetry in critical organs with Ac-225-PSMA-617 and other PSMA-radionuclide therapies.

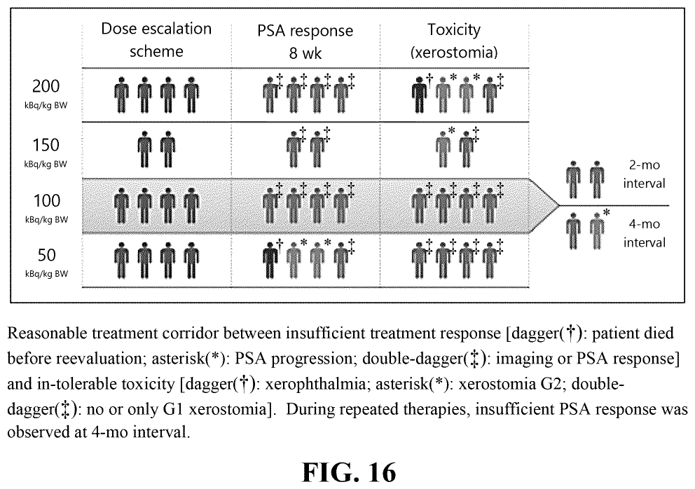

FIG. 16 depicts a summary of the outcome of the dose finding of Ac-225-PSMA.

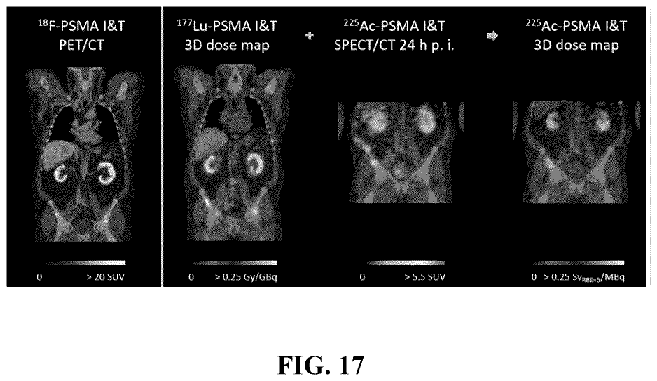

FIG. 17 depicts a summary of dosimetric γ-imaging for Ac-225-PSMA with Lu-177

### DETAILED DESCRIPTION OF THE INVENTION

Disclosed herein is a small molecular inhibitor of PSMA that has the desirable attributes of large monoclonal antibodies with reduced negative aspects, e.g., poor permeability and toxicity. The radiopharmaceutical composition disclosed herein comprises actinium-225. ²²⁵Ac-PSMA I&T is a short-lived radiolabeled substance from which the product is formulated immediately after finished synthesis.

Headings included herein are simply for ease of reference and are not intended to limit the disclosure in any way.

### I. Definitions

Compounds useful in the compositions and methods include those described herein in any of their pharmaceutically acceptable forms, including isomers such as diastereomers and enantiomers, salts, solvates, and polymorphs, as well as racemic mixtures and pure isomers of the compounds described herein, where applicable.

When introducing elements of the various embodiment(s) of the present disclosure, the articles “a”, “an”, “the” and “said” are intended to mean that there are one or more of the elements. The terms “comprising”, “including” and “having” are intended to be inclusive and mean that there may be additional elements other than the listed elements.

The use of individual numerical values are stated as approximations as though the values were preceded by the word “about” or “approximately.” Similarly, the numerical values in the various ranges specified in this application, unless expressly indicated otherwise, are stated as approximations as though the minimum and maximum values within the stated ranges were both preceded by the word “about” or “approximately.” In this manner, variations above and below the stated ranges can be used to achieve substantially the same results as values within the ranges. As used herein, the terms “about” and “approximately” when referring to a numerical value shall have their plain and ordinary meanings to a person of ordinary skill in the art to which the disclosed subject matter is most closely related or the art relevant to the range or element at issue. The amount of broadening from the strict numerical boundary depends upon many factors. For example, some of the factors which may be considered include the criticality of the element and/or the effect a given amount of variation will have on the performance of the claimed subject matter, as well as other considerations known to those of skill in the art. As used herein, the use of differing amounts of significant digits for different numerical values is not meant to limit how the use of the words “about” or “approximately” will serve to broaden a particular numerical value or range. Thus, as a general matter, “about” or “approximately” broaden the numerical value. Also, the disclosure of ranges is intended as a continuous range including every value between the minimum and maximum values plus the broadening of the range afforded by the use of the term “about” or “approximately.” Consequently, recitation of ranges of values herein are merely intended to serve as a shorthand method of referring individually to each separate value falling within the range, unless otherwise indicated herein, and each separate value is incorporated into the specification as if it were individually recited herein.

The term “CRPC,” as used herein, refers to castrate serum testosterone<50 μg/l or 1.7 nmol/l plus one of the following types of progression: biochemical progression or radiologic progression, as defined below.

The term “biochemical progression,” as used herein, refers to three consecutive rises in PSA one week apart, resulting in two 50% increases over the nadir, and PSA>2 μg/l.

The term “radiologic progression,” as used herein, refers to the appearance of new lesions; either two or more new bone lesions on bone scan or a soft tissue lesion using the Response Evaluation Criteria in Solid Tumors (RECIST).

The term “PSMA,” as used herein, refers to prostate-specific membrane antigen (PSMA), also known as folate hydrolase I or glutamate carboxypeptidase II, and is a type II transmembrane protein, which is anchored in the cell membrane of prostate epithelial cells. PSMA is highly expressed on prostate epithelial cells and strongly up-regulated in prostate cancer. The PSMA expression levels are directly correlated to androgen independence, metastasis, and prostate cancer progression. Thus, PSMA is a promising molecular target for diagnosis and therapy of metastatic prostate cancer at present.

The term “Actinium-225 (²²⁵Ac),” as used herein, refers to Actinium-225 (²²⁵Ac), an alpha emitter, which has been labelled to PSMA ligands as ²²⁵Ac-PSMA for targeted alpha therapy (TAT). ²²⁵Ac has a half-life of 9.9 days and decays to produce four α-particles with an energy of 5.8-8.4 MeV, with a tissue range of up to 85 μm. Alpha particles are attractive anti-tumor agents as they have a high linear energy transfer (LET) and relatively short tissue length and are able to produce double-strand DNA damage whilst minimizing toxicity to adjacent tissue, this is a far more favorable cytotoxic agent as compared to p particle emission which mainly results in single strand DNA breaks and a relatively long tissue path length which contributes to its toxicity profile.

The term “PSMA-617,” as used herein, refers to a DOTA derivative of the Glu-urea-Lys motif that has been developed in the German Cancer Research Center (DKFZ) Heidelberg, Germany, for the treatment of patients with metastatic prostate cancer.

The term “PSMA-I&T,” as used herein, refers to ²²⁵Ac-PSMA for imaging and therapy (I&T), a third-generation derivative of PSMA-compounds which has been used here. It is a synonym for DOTAGA-(1-y)fk(Sub-KuE).

The term “(P)RLT,” as used herein, refers to (Prostate) radioligand therapy, which in this context involves the systemic intravenous administration of a specific radiopharmaceutical composed of α-emitting or β-emitting radionuclide chelated to a small molecule for the purpose of delivering cytotoxic radiation to cancer cells.

The term “relative biological effectiveness (RBE),” as used herein, refers to the ratio of biological effectiveness of one type of ionizing radiation relative to another, given the same amount of absorbed energy: here β- and α-emission, between the ¹⁷⁷Lu and ²²⁵Ac (as -biological consequence of different ionisation-densities along a particle-tract). The RBE is an empirical value that varies depending on the type of ionizing radiation, the energies involved, the biological effects being considered such as cell death, and the oxygen concentration etc. RBE was 5 (for ²²⁵Ac/¹⁷⁷Lu) in an experimental neuroendocrine tumor model.

The term “half-life” as used herein, refers to the time required for a drug's blood or plasma concentration to decrease by one half. This decrease in drug concentration is a reflection of its excretion or elimination after absorption is complete and distribution has reached an equilibrium or quasi equilibrium state. The half-life of a drug in the blood may be determined graphically off of a pharmacokinetic plot of a drug's blood-concentration time plot, typically after intravenous administration to a sample population. The half-life can also be determined using mathematical calculations that are well known in the art. Further, as used herein the term “half-life” also includes the “apparent half-life” of a drug. The apparent half-life may be a composite number that accounts for contributions from other processes besides elimination, such as absorption, reuptake, or enterohepatic recycling.

The term “active agent” or “drug,” as used herein, refers to any chemical that elicits a biochemical response when administered to a human or an animal. The drug may act as a substrate or product of a biochemical reaction, or the drug may interact with a cell receptor and elicit a physiological response, or the drug may bind with and block a receptor from eliciting a physiological response.

The terms “subject” or “patient” are used interchangeably herein and refer to a vertebrate, preferably a mammal. Mammals include, but are not limited to, humans.

### II. Radiopharmaceutical Compositions Comprising Actinium

[²²⁵Ac]Actinium-PSMA-I&T is also known by its synonyms as follows: ²²⁵Ac-ITG-PSMA-1 or ²²⁵Ac-PSMA-TUM3 or ²²⁵Ac-DOTAGA-(I-y)fk(Sub-KuE) or (3S,7S,26R,29R,32R,37R)-29-benzyl-32-(4-hydroxy-3-iodobenzyl)-5,13,20,28,31,34-hexaoxo-37-(4,7,10-tris(carboxymethyl)-1,4,7,10-tetraazacyclododecan-1-yl)-4,6,12,21,27,30,33-heptaazaheptatriacontane-1,3,7,26,37-pentacarboxylic acid; Actinium-225 (III). The molecular formula of the unlabeled precursor is C₆₃H₉₂IN₁₁O₂₃ 4TFA 3 H₂O With a relative molecular mass of 1498 g/mo1.

The labelled-substance; ²²⁵Ac-PSMA I&T is labelled with Actinium-225 (T_{1/2}=9,9d) solution. The synthesis of ²²⁵Ac-PSMA I&T may be carried out in a TRACERlab MX synthesis module. The unit may be an automated synthesis module in a controlled environment, used for the radiolabelling. The labelling solution containing Actinium-225 nitrate (²²⁵Ac(NO₃)₃) in 0.04 M hydrochloric acid (HCl) was connected to the synthesis cassette containing other chemicals and components required for the labelling process. The ²²⁵Ac solution was rinsed with 0.04 M HCl and transferred to the synthesis cassette reactor where it was mixed with 0.04 M sodium ascorbate solution containing PMSA I&T precursor. The resultant solution was heated in the reactor and after heating the produced ²²⁵Ac-PSMA I&T was trapped into a pre-conditioned (EtOH)C-18 cartridge. The cartridge was rinsed with sterile water and the final product was eluted from the C-18 cartridge with 1.5 ml of 50% sterile ethanol into a bulk vial. For formulation purposes 8.5 ml of a formulation matrix containing 50 mg/ml ascorbic acid was added to the bulk vial to achieve a final product volume of 10 ml and radioactivity concentration from about 0.2 MBq/ml to about 1.5 MBq/ml.

In one embodiment, the final product may have a radioactivity concentration of about 0.3 MBq/ml to about 1.4 MBq/ml. In another embodiment, the final product may have a radioactivity concentration of about 0.4 MBq/ml to about 1.3 MBq/ml. In still another embodiment, the final product may have a radioactivity concentration of about 0.5 MBq/ml to about 1.3 MBq/ml. In yet another embodiment, the final product may have a radioactivity concentration of about 0.6 MBq/ml to about 1.1 MBq/ml. In still other embodiments, the final product may have a radioactivity concentration of about 0.2 MBq/ml, about 0.3 MBq/ml, about 0.4 MBq/ml, about 0.5 MBq/ml, about 0.6 MBq/ml, about 0.7 MBq/ml, about 0.8 MBq/ml, about 0.9 MBq/ml, about 1.0 MBq/ml, about 1.1 MBq/ml, about 1.2 MBq/ml, about 1.3 MBq/ml, about 1.4 MBq/ml, or about 1.5 MBq/ml.

The synthetized ²²⁵Ac-PSMA I&T solution is formulated in an injections grade water solution containing stabilizing agents. The solution is sterilized by aseptic filtration through a 0.22 μm filter prior to dispensing in multidose vials. Administration of the formulated solution is within 48 h of the end of the synthesis after quality control and of the drug product.

The drug product has a shelf life at temperatures ranging from 2° C.-25° C. The drug product also meets the requirements for sterility and bacterial endotoxins according to the European pharmacopoeia confirming an acceptable manufacturing process from a microbial point of view.

Due to the highly atypical properties of the ²²⁵Ac labelled molecule (short half-life and picomolar quantities synthesized), a mass spectroscopy-based approach is not feasible to accomplish in practice. Instead an indirect approach for structural confirmation of ²²⁵Ac-PSMA I&T is used.

As Actinium does not have a stable form, a Lu-labelled peptide has been used to verify the structure of the labelled precursor. As Lutetium has very similar chemical characteristics and both a stable and a radioactive form, it has been used as a reference standard for the identification method.

Immediately after production, a sample of ²²⁵Ac-PSMA I&T solution is spotted onto a TLC plate which is then developed and analysed using a High Purity Germanium (HPGe) Radiation Detector. A ¹⁷⁷Lu-PSMA I&T is used as a reference standard. The ¹⁷⁷Lu-PSMA I&T has been eluted against a ^{nat}Lu-PSMA I&T standards on an analytical high-performance liquid chromatography (HPLC).

The medicinal product is a sterile filtered radiopharmaceutical solution containing a micro dose of ²²⁵Ac-PSMA I&T solution in a 42.5 mg/ml aqueous ascorbic acid solution containing 59 mg/ml ethanol. The product is diluted to a standard concentration and therefore the final volume of the bulk product varies depending on the starting activity introduced. The composition of the final product is described below in Table 1:

TABLE 1
Composition of final product*

| Component | Quantity | | Function |
|---|---|---|---|
| ²²⁵Ac-PSMA-I&T | q.s.** | | API |
| Ethanol | 59 | mg | Vehicle/Stabilizing agent |
| | | | (radiolysis) |
| Ascorbic acid | 42.5 | mg | Stabilizing agent (radiolysis) |
| Disodium EDTA | 21 | μg | Metal ion chelator |

| | | |
|---|---|---|
| Sodium bicarbonate | q.s. | pH adjuster |
| Sodium hydroxide | q.s. | pH adjuster |
| WFI (injections grade water) | ad 1 ml | Vehicle |

*Max volume per vial is 10 ml
**sufficient amount of radioactivity for intended use

In another embodiment, the total amount of ²²⁵Ac-PSMA-I&T present in the radiopharmaceutical composition can and will vary. In some embodiments, the total amount of ²²⁵Ac-PSMA-I&T present in the radiopharmaceutical composition may range from about 9 μg/ml to 20 μg/ml, 10 μg/ml to 20 μg/ml, 11 μg/ml to 20 μg/ml, 11 μg/ml to 15 μg/ml, 11 μg/ml to 14 μg/ml, or 11 μg/ml to 13 μg/ml. In another embodiment, the total amount of ²²⁵Ac-PSMA-I&T in the radiopharmaceutical composition may range from about 5 μg/ml to about 15 μg/ml. In various embodiments, the total amount of ¹²²⁵Ac-PSMA-I&T present in the radiopharmaceutical composition may be about 5 μg/ml, 6 μg/ml, 7 μg/ml, 8 μg/ml, 9 μg/ml, 10 μg/ml, 11 μg/ml, 12 μg/ml, 13 μg/ml, 14 μg/ml, 15 μg/ml, 16 μg/ml, 17 μg/ml, 18 μg/ml, 19 μg/ml, or 20 μg/ml.

The total amount of ethanol present in the radiopharmaceutical composition can and will vary. In some embodiments, the total amount of ethanol present in the radiopharmaceutical composition may range from about 40 mg to 120 mg, about 50 to 90 mg, about 50 to 80 mg, or about 60 to 80 mg. In another embodiment, the total amount of ethanol in the radiopharmaceutical composition may range from about 65 mg to about 80 mg. In various embodiments, the total amount of ethanol present in the radiopharmaceutical composition may be about 40 mg, 45 mg, 50 mg, 55 mg, 60 mg, 65 mg, 70 mg, 71 mg, 72 mg, 73 mg, 74 mg, 75 mg, 76 mg, 77, mg, 78, mg, 79 mg, 80 mg, 85 mg, 90 mg, 95 mg, 100 mg, 105 mg, 110 mg, 115 mg, or 120 mg. In one embodiment, the total amount of ethanol in the radiopharmaceutical composition may be about 52 mg. In another embodiment, the total amount of ethanol in the radiopharmaceutical composition may be about 59 mg. In yet another embodiment, the total amount of ethanol in the radiopharmaceutical composition may be about 68 mg. In a further embodiment, the ratio of ethanol in the radiopharmaceutical composition may be about 53 mg per 10 ml. In another embodiment, the ratio of ethanol in the radiopharmaceutical composition may be about 59 mg per 10 ml. In still another embodiment, the ratio of ethanol in the radiopharmaceutical composition may be about 61 mg per 10 ml.

The total amount of ascorbic acid in the radiopharmaceutical composition can and will vary. In some embodiments, the total amount of ascorbic acid present in the radiopharmaceutical composition may range from about 20 mg to 90 mg, about 20 to 80 mg, about 20 to 70 mg, about 20 to 60 mg, about 20 to 50 mg, about 25 to 50 mg, about 30 to 50 mg or about 35 to 45 mg. In another embodiment, the total amount of ascorbic acid in the radiopharmaceutical composition may range from about 5 mg to about 50 mg. In various embodiments, the total amount of ascorbic acid present in the radiopharmaceutical composition may be about 5 mg, 10 mg, 15 mg, 20 mg, 25 mg, 30 mg, 35 mg, 36 mg, 37 mg, 38 mg, 39 mg, 40 mg, 40.5 mg, 41 mg, 41.5 mg, 42 mg, 42.5 mg, 43 mg, 43.5 mg, 44 mg, 44.5 mg, 45 mg, 50 mg, 55 mg, 60 mg, 65 mg, 70 mg, 75 mg, 80 mg, 85 mg, or 90 mg. In one embodiment, the total amount of ascorbic acid in the radiopharmaceutical composition may be about 41.5 mg. In another embodiment, the total amount of ascorbic acid in the radiopharmaceutical composition may be about 40.5 mg. In yet another embodiment, the total amount of ascorbic acid in the radiopharmaceutical composition may be about 44.5 mg. In a further embodiment, the ratio of ascorbic acid in the radiopharmaceutical composition may be about 40.5 mg per 10 ml. In another embodiment, the ratio of ascorbic acid in the radiopharmaceutical composition may be about 42.5 mg per 10 ml. In still another embodiment, the ratio of ascorbic acid in the radiopharmaceutical composition may be about 44.5 mg per 10 ml. In yet another embodiment, the percentage of ascorbic acid concentration in the radiopharmaceutical composition may be about 10 to 80 mg/ml, 10 to 75 mg/ml, 10 to 70 mg/ml, 15 to 80 mg/ml, 15 to 75 mg/ml, 15 to 70 mg/ml, 20 to 80 mg/ml, 20 to 75 mg/ml, or 20 to 70 mg/ml.

In some embodiments, the disclosure provides for a radiopharmaceutical composition with a micro dose of ²²⁵Ac-PSMA I&T solution and at least metal ion chelator. A suitable chelating agent may include ethylenediamine tetracetic acid (EDTA) and its salts, N-(hydroxy-ethyl)ethylenediaminetriacetic acid, nitrilotriacetic acid (NIA), ethylene-bis(oxyethylene-nitrilo)tetraacetic acid, 1,4,7,10-tetraazacyclodo-decane-N,N′,N″,N″′-tetraacetic acid, 1,4,7,10-tetraaza-cyclododecane-N,N′,N″-triacetic acid, 1,4,7-tris(carboxymethyl)-10-(2′-hydroxypropyl)-1,4,7,10-tetraazocyclodecane, 1,4,7-triazacyclonane-N,N′,N″-triacetic acid, 1,4,8,11-tetraazacyclotetra-decane-N,N′,N″,N′″-tetraacetic acid; diethylenetriamine-pentaacetic acid (DTPA), ethylenedicysteine, bis(aminoethanethiol)carboxylic acid, triethylenetetraamine-hexaacetic acid, and 1,2-diaminocyclohexane-N,N,N′,N′-tetraacetic acid. In one embodiment, the chelating agent may be the sodium salt of EDTA. The amount of chelating agent present in the radiopharmaceutical composition may range from about 5 μg to 50 μg. In some embodiments, the amount of chelating agent present may be about 5 μg, 6 μg, 7 μg, 8 μg, 9 μg, 10 μg, 11 μg, 12 μg, 13 μg, 14 μg, 15 μg, 16 μg, 17 μg, 18 μg, 19 μg, 20 μg, 21 μg, 22 μg, 23 μg, 24 μg, 25 μg, 26 μg, 27 μg, 28 μg, 29 μg, 30 μg, 31 μg, 32 μg, 33 μg, 34 μg, 35 μg, 40 μg, 45 μg, or 50 μg. In another embodiment, the amount of chelating agent present may be from about 0.001% to about 0.20% (w/w) of such radiopharmaceutical composition. In some embodiments, the amount of chelating agent present in a radiopharmaceutical composition may be about 0.001%, 0.002%, 0.003%, 0.004%, 0.005%, 0.01%, 0.02%, 0.03%, 0.04%, 0.05%, 0.06%, 0.07%, 0.08%, 0.09%, 0.10%, 0.11%, 0.12%, 0.13%, 0.14%, or 0.15% (w/w) of the total weight of the radiopharmaceutical composition.

One aspect of the disclosure provides for a radiopharmaceutical composition with a pH of about 3 to 9, 4 to 9, 5 to 9, 3 to 8, 4 to 8, or 5 to 8. The pH of the radiopharmaceutical composition may be about 4, 4.5, 4.6. 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2. 7.3, 7.4. 7.5, 7.6. 7.7, 7.8, 7.9, 8.0, 8.1, 8.2, 8.3, 8.4, 8.5, or 9.

Another aspect of the disclosure provides for a radioactive content of about 70% to 130%. The radioactive content of the radiopharmaceutical composition may be about 70% to 125%, 70% to 120%, 70% to 115%, 70% to 110%, 80% to 130%, 85% to 130%, 90% to 130%, 95% to 130%, 75% to 125%, 75% to 120%, 75% to 115%, 75% to 110%, 80% to 125%, 80% to 120%, 80% to 115%, 80% to 110%, 85% to 125%, 85% to 120%, 85% to 115%, 85% to 110%, 90% to 125%, 90% to 120%, 90% to 115%, or 90% to 110%.

The synthesis is a one-step labelling process with injections grade ethanol and water used as the only solvents, therefore no residual solvents are present. Radiochemical impurities are quantified by chromatographical methods (TLC/HPGe). Radiochemical purity must not be less than 95.0% in total. Chemical impurities are quantified by chromatographical methods (HPLC).

²²⁵Ac-PSMA I&T is a relatively short-lived radiolabelled substance from which the product is formulated immediately after finished synthesis. Therefore, there are no specifications or batch analysis results for the labelled substance. Controls are performed on the labelled drug product.

A precursor standard manufactured by piCHEM Forschungs-und Entwicklungs GmbH (piCHEM) is used for quantification of the unlabelled/unreacted PSMA I&T precursor and non-radioactive metal chelates of PSMA I&T. The UV-absorption of metal chelates of PSMA I&T is very similar to that of the unlabelled precursor at the UV-wavelength used (280 nm) and is therefore considered suitable for quantification of trace amounts of metal chelates of PSMA I&T that may be co-produced during the heated complexation reaction of ²²⁵Ac and PSMA I&T.

A stable reference standard for ^{nat} Lu-PSMA I&T manufactured by piCHEM is used for verifying HPLC system suitability prior to ²²⁵Ac-PSMA I&T sample analysis.

The formulation solution is prepared from an injections grade solution containing ascorbic acid (Ascor L) injections grade water.

Specifications for the ²²⁵Ac-Lu-PSMA-I&T solution are presented in Table 2 below. The specifications listed are used as release parameters except for sterility testing. Sterility is tested on all batches post-release.

TABLE 2
Specifications for ²²⁵Ac-Lu-PSMA-I&T

| Characteristics | Method | Specification |
|---|---|---|
| Appearance | Visual Inspection | Clear, colorless or yellow liquid, no |
| | | visible particles |
| pH | Ph. Eur. | 5.0-8.0 |
| Ascorbic acid concentration | Colorimetric strip | 20-70 mg/ml |
| | test | |
| Identification test A | MCA | ²²⁵Ac daughter nuclide main peak |
| | | gamma-energy: |
| | | ²²¹Fr: 218.19 ± 4.36 keV (± 2%, HPGe) |
| | | ²¹³Bi: 440.46 ± 8.81 keV (± 2%, HPGe) |
| Identification test B | TLC | Retention of ²²⁵Ac-PSMA I&T is |
| | | comparable to ¹⁷⁷Lu-PSMA-I&T on a |
| | | TLC method |
| Radiochemical purity (TLC) | TLC | ²²⁵Ac-PSMA I&T radioactivity ≥ 95% |
| | | from total radioactivity at the end of |
| | | shelf life |
| Chemical purity (HPLC) | HPLC | PSMA-I&T concentration ≤ 12.0 μg/mL |
| | | Other impurities in total ≤ 12.0 μg/mL |
| Bacterial Endotoxins | Ph. Eur. | <17.5 IU/ml (≤175 IU/Dose) |
| Radioactive content | MCA | 90%-110% at the date and time stated |
| | | on the label |
| Sterility | Ph. Eur. | Sterile |
| Sterile filter integrity test | Bubble Point | ≥3, 32 bar, (water, PALL Mini |
| | | Kleenpak) |
| Expiry | Stability Study | 48 h from end of synthesis |
| Maximum fill volume | | ≤10 mL |

a. Analytical Procedures

The product is identified by subsequent injection of reference solution of Lu-PSMA-I&T and formulated solution to a liquid chromatography system. Radio nuclidic identity is determined by gamma ray energy detection.
pH is estimated by pH paper.
Radioactivity is measured in a dose calibrator
Radiochemical purity is determined by liquid chromatography with radioactivity detection and thin layer chromatography

The chemical amount of [²²⁵Ac]-Lu-PSMA-I&T is calculated from the radioactivity measurement when the dose is dispensed.

Bacterial endotoxin content is determined for each batch before release using a PTS-tester (Ph Eur method D).
Sterility is determined according to Ph Eur

The analytical procedures used for the drug product—e.g., specificity, linearity, and reproducibility-were investigated by using a known amount of reference standards for the unlabelled precursor. All analytical procedures were found suitable for their intended use.

An acceptance criterion for the amount of radioactivity in the formulation is not set since this will vary depending on the individual clinical need assessed by the health care professional responsible for administering the formulation. The radioactive content must be within 90%-110% of the stated value at the date and time stated on the label.

b. Stability Data for the Radiopharmaceutical Composition

The radiochemical purity and chemical properties of the formulated solution (pH, impurities, visual properties) were tested on the three batches over a time span of 48 hours from the end-of-synthesis time. All batches were stored in room temperature and one batch in stress conditions (+2-8° C. and +40° C.) and tested at 24 h and 48 h after EOS. No deviations from specifications were observed. Based on these results a shelf life of 48 hours from EOS is justified at storage temperatures below 25° C.

The drug substance is immediately formulated as a step in the automated synthesis process and stability studies are available for the prepared medicinal product (see Table 3 below).

TABLE 3
Stability Data: Chemical quality at 48 h post end of synthesis

| | | | | | Stability data at 48 h after end of synthesis | | | | | |
|---|---|---|---|---|---|---|---|---|---|---|
| Stability criteria | | | | | ACMI2115D-1, | | ACMI2115D-1, | | ACMI2115D-1, | |
| Test | Specification | | ACMI2114B-1 | | +5° C. | | +20° C. | | +40° C. | |
| Visual inspection | Clear, colorless or | | Pass | | Pass | | Pass | | Pass | |
| | slightly yellow liquid, | | | | | | | | | |
| | no visible particles | | | | | | | | | |
| pH | 5.0-8.0 | | 5.5 | | 6.0 | | 5.0 | | 5.0 | |
| ²²¹Fr and ²¹³Bi | ²²¹Fr: 218 ± 2 keV | | ²²¹Fr: 218 keV | | ²²¹Fr: 218 kev | | ²²¹Fr: 218 keV | | ²²¹Fr: 218 ke V | |
| main peak | ²¹³Bi: 440 ± 4 keV | | ²¹³Bi: 441 Kev | | ²¹³Bi: 441 keV | | ²¹³Bi: 441 keV | | ²¹³Bi: 441 keV | |
| gammaenergy | (HPGe-detector) | | | | | | | | | |
| Radioactivity | No specification set | | 0.820 | MBq/ml | 1.000 | MBq/ml | 1.009 | MBq/ml | 0.996 | MBq/ml |
| concentration | | | 08.04.2021 10:19 | | 17.04.2021 10:10 | | 17.04.2021 9:51 | | 17.04.2021 10:25 | |
| (MBq/ml) | | | | | | | | | | |
| Radiochemical purity | ²²⁵Ac-PSMA I&T | | 96.1% | | 95.2% | | 94.7% | | 89.6% | |
| % (TLC/HPGe) | radioactivity ≥ | | | | | | | | | |
| | 95% from total | | | | | | | | | |
| | radioactivity | | | | | | | | | |
| Chemical purity | <12.0 | μg/ml | 2.0 | μg/ml | 8.1 | μg/ml | 6.4 | μg/ml | 6.3 | μg/ml |
| PSMA-I&T | | | | | | | | | | |
| Chemical purity | <12.0 | μg/ml | 8.7 | μg/ml | 6.3 | μg/ml | 6.4 | μg/ml | 8.4 | μg/ml |
| Other impurities | | | | | | | | | | |
| Specific activity | No specification set | | 0.115 | MBq/nmol | 0.104 | MBq/nmol | 0.118 | MBq/nmol | 0.101 | MBq/nmol |
| Ascorbic acid | 20-70 | mg/ml | 30 | mg/ml | 30 | mg/ml | 30 | mg/ml | 30 | mg/ml |

| Stability criteria | | | Stability data at 48 h after end of synthesis | | | | | |
|---|---|---|---|---|---|---|---|---|
| Test | Specification | | ACMI2115D-2 | | ACMI2119B-1 | | ACMI2119E-1 | |
| Visual inspection | Clear, colorless or | | Pass | | Pass | | Pass | |
| | slightly yellow liquid, | | | | | | | |
| | no visible particles | | | | | | | |
| pH | 5.0-8.0 | | 5.5 | | 5.5 | | 5.5 | |
| ²²¹Fr and ²¹³Bi | ²²¹Fr: 218 ± 2 keV | | ²²¹Fr: 218 keV | | ²²¹Fr: 218 keV | | ²²¹Fr: 218 keV | |
| main peak | ²¹³Bi: 440 ± 4 keV | | ²¹³Bi: 441 keV | | ²¹³Bi: 441 keV | | ²¹³Bi: 441 keV | |
| gammaenergy | (HPGe-detector) | | | | | | | |
| Radioactivity | No specification set | | 0.957 | MBq/ml | 0.901 | MBq/ml | 0.915 | MBq/ml |
| concentration | | | 17.04.2021 10:38 | | 13.05.2021 11:15 | | 16.05.2021 9:19 | |
| (MBq/ml) | | | | | | | | |
| Radiochemical purity | ²²⁵Ac-PSMA I&T | | 95.7% | | 94.8% | | 95.2% | |
| % (TLC/HPGe) | radioactivity ≥ | | | | | | | |
| | 95% from total | | | | | | | |
| | radioactivity | | | | | | | |
| Chemical purity | <12.0 | μg/ml | 5.6 | μg/ml | 5.1 | μg/ml | 4.6 | μg/ml |
| PSMA-I&T | | | | | | | | |
| Chemical purity | <12.0 | μg/ml | 6.4 | μg/ml | 7.8 | μg/ml | 7.7 | μg/ml |
| Other impurities | | | | | | | | |
| Specific activity | No specification set | | 0.119 | MBq/nmol | 0.105 | MBq/nmol | 0.112 | MBq/nmol |
| Ascorbic acid | 20-70 | mg/ml | 50 | mg/ml | 30 | mg/ml | 30 | mg/ml |

b. Process Validation

All validation batches were tested and confirmed to be sterile. The sterility test was validated on batches ACMI2119E-1, ACMI2119E-2, ACMI2123E-1, ACMI2123E-2 and ACMI2123E-3. Two sets of validation were performed from batch ACMI2119E-2. Batches ACMI2123E-1, ACMI2123E-2 and ACMI2123E-3 were produced as additional batches to confirm growth of a control microbe (Candida albicans) that did not show any growth in one of the control samples from batch ACMI2119E-2. Based on the results, no microbial inhibition was observed on any tested microbe and the observation for C. albicans on one sample was assessed to have been caused by a laboratory error.

The integrity of sterile filters were tested on all validation batches and all results conformed to specifications.

All validation batches where tested for endotoxins and conformed to specifications.

The process bioburden was tested at three batches and none showed microbiological growth over the detection limit. Bioburden sample was produced by from batches ACMI2120E-1, ACMI2121A-1 and ACMI2121A-2 by removing the 0.22 um filter from the dispensing process. The test was validated using the same batches. All three bioburden batches were also tested for chemical quality and complied to all specifications.

Altogether, it is concluded that the manufacturing process reliably produces ²²⁵Ac-PSMA I&T solution in satisfactory amounts and quality for clinical use under supervision of qualified healthcare professionals. Based on the results from the stability study the product needs to be stored at 5° C.-25° C. to maintain stability of 48 h.

TABLE 4a
Chemical quality of validation batches

| SYNTHESIS INFORMATION | | | | | | | | |
|---|---|---|---|---|---|---|---|---|
| BATCH ID | | | ACMI2114B-1 | | ACMI2115D-1 | | ACMI2115D-2 | |
| Ac-225 | | | Order No. 11045881 | | Order No. 11046459 | | Order No. 11046460 | |
| Ac-225 Activity (MBq) | | | 21 | MBq | 19 | MBq | 19 | MBq |
| | | | 31.03.2021 12:00 * | | 12.04.2021 12:00 * | | 12.04.2021 12:00 * | |
| Start of synthesis | | | 06.04.2021 09:22:24 | | 15.04.2021 08:13:15 | | 15.04.2021 09:02:38 | |
| End of synthesis | | | 06.04.2021 09:49:21 | | 15.04.2021 08:40:13 | | 15.04.2021 09:29:35 | |
| QC TESTING | | | | | | | | |
| Validation criteria | | | | | | | | |
| Test | Specification | | | | | | | |
| Visual inspection | Clear, colorless or slightly yellow | | Pass | | Pass | | Pass | |
| | liquid, no visible particles | | | | | | | |
| pH | 5.0-8.0 | | 5.5 | | 6.0 | | 6.0 | |
| ²²¹Fr ja ²¹³Bi main peaks | ²²¹Fr: 218 ± 2 keV | | ²²¹Fr: 218 keV | | ²²¹Fr: 218 keV | | ²²¹Fr: 218 keV | |
| gammaenergy | ²¹³Bi: 440 ± 4 keV | | ²¹³Bi: 441 keV | | ²¹³Bi: 440 keV | | ²¹³Bi: 440 keV | |
| | (HPGe-detector) | | | | | | | |
| Radioactivity | No specification set | | 0.895 | MBq/ml | 1.041 | MBq/ml | 1.121 | MBq/ml |
| concentration (MBq/ml) | | | 07.04.2021 07:47 | | 16.04.2021 09:00 | | 16.04.2021 13:32 | |
| Radiochemical purity % | ²²⁵Ac-PSMA I&T radioactivity ≥ | | 96.5% | | 95.3% | | 96.6% | |
| (TLC/HPGe) | 95% from total radioactivity | | | | | | | |
| Chemical purity | <12.0 | μg/ml | 3.2 | μg/ml | 7.5 | μg/ml | 7.7 | μg/ml |
| PSMA I&T | | | | | | | | |
| Chemical purity | <12.0 | μg/ml | 9.1 | μg/ml | 6.1 | μg/ml | 6.5 | μg/ml |
| Other impurities | | | | | | | | |
| Specific activity | No specification set | | 0.109 | MBq/nmol | 0.114 | MBq/nmol | 0.118 | MBq/nmol |
| Radionuclidic purity | ≥99.9% | | ≥99.9% | | ≥99.9% | | ≥99.9% | |
| Ascorbic acid | 20-70 | mg/ml | 50 | mg/ml | 50 | mg/ml | 50 | mg/ml |

| | | | | | |
|---|---|---|---|---|---|
| Bacterial endotoxins | <17.5 | (EU/ml) | <5.00 | <5.00 | <5.00 |

| Filter integrity test | Bubble point > 3.45 bar (water) | 3.87 | 4.16 | 3.97 |
|---|---|---|---|---|
| Sterility | Sterile | Sterile | Sterile | Sterile |

TABLE 4b
Chemical quality of validation batches

| SYNTHESIS INFORMATION | | | | | | | | |
|---|---|---|---|---|---|---|---|---|
| BATCH ID | | | ACMI2119B-1 | | ACMI2119E-1 | | ACMI2119E-2 | |
| Ac-225 | | | Order No. 11047251 | | Order No. 11047252 | | Order No. 11047253 | |
| Ac-225 Activity (MBq) | | | 16 | MBq | 17 | MBq | 10 | MBq |
| | | | 10.05.2021 12:00 * | | 12.05.2021 12:00 * | | 12.05.2021 12:00 * | |
| Start of synthesis | | | 11.05.2021 08:14:40 | | 14.05.2021 08:22:06 | | 14.05.2021 09:02:00 | |
| End of synthesis | | | 11.05.2021 08:42:04 | | 14.05.2021 08:49:30 | | 14.05.2021 09:29:25 | |
| QC TESTING | | | | | | | | |
| Validation criteria | | | | | | | | |
| Test | Specification | | | | | | | |
| Visual inspection | Clear, colorless or | | Pass | | Pass | | Pass | |
| | slightly yellow liquid, | | | | | | | |
| | no visible particles | | | | | | | |
| pH | 5.0-8.0 | | 5.5 | | 5.5 | | 5.5 | |
| ²²¹Fr ja ²¹³Bi main | ²²¹Fr: 218 ± 2 keV | | ²²¹Fr: 218 keV | | ²²¹Fr: 218 keV | | ²²¹Fr: 218 keV | |
| peaks | ²¹³Bi: 440 ± 4 keV | | ²¹³Bi: 441 keV | | ²¹³Bi: 440 keV | | ²¹³Bi: 440 keV | |
| gammaenergy | (HPGe-ilmaisin) | | | | | | | |
| Radioactivity | No specification set | | 0.935 | MBq/ml | 0.917 | MBq/ml | 0.561 | MBq/ml |
| concentration | | | 12.05.2021 08:58 | | 16.05.2021 07:54 | | 16.05.2021 08:09 | |
| (MBq/ml) | | | | | | | | |
| Radiochemical | ²²⁵Ac-PSMA I&T | | 95.5% | | 96.1% | | 96.2% | |
| purity % | radioactivity ≥ | | | | | | | |
| (TLC/HPGe) | 95% from total | | | | | | | |
| | radioactivity | | | | | | | |
| Chemical purity | <12.0 | μg/ml | 7.1 | μg/ml | 6.6 | μg/ml | 6.6 | μg/ml |
| PSMA I&T | | | | | | | | |
| Chemical purity | <12.0 | μg/ml | 4.3 | μg/ml | 6.3 | μg/ml | 5.7 | μg/ml |
| Other impurities | | | | | | | | |
| Specific activity | No specification set | | 0.122 | MBq/nmol | 0.107 | MBq/nmol | 0.068 | MBq/nmol |
| Radionuclidic | ≥99.9% | | ≥99.9% | | ≥99.9% | | ≥99.9% | |
| purity | | | | | | | | |
| Ascorbic acid | 20-70 | mg/ml | 30 | mg/ml | 30 | mg/ml | 30 | mg/ml |

| | | | | | |
|---|---|---|---|---|---|
| Bacterial | <17.5 | (EU/ml) | <5.00 | <5.00 | <5.00 |

| endotoxins | | | | |
|---|---|---|---|---|
| Filter integrity test | Bubble point > 3.45 | 3.87 | 3.99 | 3.96 |
| | bar (water) | | | |

| | | | | |
|---|---|---|---|---|
| Sterility | Sterile | Sterile | Sterile | Sterile |

c. Clinical Experience in ²²⁵Ac-PSMA

Bismuth-213 for PSMA-targeted α-therapy (TAT) is a mixed α- and β-emitter with a half-life of 45.6 min and it is produced from Ac-225 decay (FIG. 2). Small molecule PSMA-I&T induced more double-strand breaks than the nanobody in nonclinical studies, where targeted α-therapy with ²¹³Bi labeled antibody (J591), small molecule inhibitor PSMA-I&T or nanobody (JVZ-008) were compared; they demonstrated tumor targeting and tumor growth inhibition in nude mice with PSMA-overexpressing xenografts. Dosimetry calculations with ²¹³Bi-PSMA-617 and ²²⁵Ac-PSMA-617 demonstrated the superiority of ²²⁵Ac as compared to short-lived ²¹³Bi as the radionuclide label for PSMA-617. Probably therefore, there is only one single patient case reported to date on the use of ²¹³Bi-PSMA-617. The patient was treated with two cycles of ²¹³Bi-PSMA-617 with a cumulative activity of 592 MBq. The serum PSA concentration decreased from 237 μg/l down to 43 μg/l as sign of biochemical response. Also, the short half-life of ²¹³Bi makes this radionuclide less suitable for routine clinical therapeutic applications.

TABLE 5
Clinical trials of Actinium-225-PSMA-targeted alpha therapy.

| | Number of | Activity per | | Biochemical | | PFS/OS | Major | |
|---|---|---|---|---|---|---|---|---|
| | patients | cycle/MBq | | response PSA₅₀ | | [mo] | toxicity | First author |
| PSMA-617 | 2 | 100 | kBq/kg | 100% | (2/2) | | xerostomia | Kratochwil |
| | | | | | | | | 2016 |
| PSMA-617 | 14 | 50-200 | kBq/kg | 44% | (4/9) | na/8.5 | xerostomia | Kratochwil |
| | | | | | | | | 2017 |
| PSMA-617 | 40 | 100 | kBq/kg | 63% | (24/38) | na/>12 | xerostomia | Kratochwil |
| | | | | | | | | 2018 |
| PSMA-617 | 1 | 8 | | 100% | (1/1) | | | Sathekge |
| | | | | | | | | 2019a |
| PSMA-617 | 17 | 4-8 | | 88% | (15/17) | | xerostomia | Sathekge |
| | | | | | | | | 2019b |
| PSMA-617 | 1 | 6-8 | | 100% | (1/1) | | xerostomia | De Medeiros |
| | | | | | | | xerophtalmia | 2019 |
| PSMA-617 | 73 | 4-8 | | 70% | (51/73) | 15.2/18.0 | | Sathekge |
| | | | | | | | | 2020 |
| PSMA-617 | 26 | 4-8 | | 65% | (17/26) | 3.5/7.7 | xerostomia | Feuerecker 2020 |
| | | | | | | | Hb↓, WBC↓, plt↓ | |
| PSMA-617 | 28 | 100 | kBq/kg | 39% | (11/28) | 12/17 | xerostomia | Yadav 2020 |
| | | | | | | | fatigue | |

| PSMA-617 | 13 | 6-8 | 69% | (9/13) | na/8.5 | xerostomia | van der Doelen |
|---|---|---|---|---|---|---|---|
| | | | | | | | 2020 |
| PSMA-I&T | 1 | 8 | 100% | (1/1) | | xerostomia | Ilhan 2020 |
| PSMA-I&T | 14 | 7.8 | 50% | (7/14) | | xerostomia | Zacherl 2021 |

²²⁵Ac has a half-life of 9.9 days and decays to produce four alpha particles with an energy of 5.8-8.4 MeV, with a tissue range of up to 85 μm. This alpha emitter has been labelled to PSMA ligands as ²²⁵Ac-PSMA for targeted alpha therapy (TAT). ²²⁵Ac deposits high energy resulting in irreparable double-strand DNA destruction whilst sparing surrounding normal tissue making it an attractive anti-tumor agent. Clinical application of ²²⁵Ac-PSMA TAT as last line of therapy in patients with mCRPC has demonstrated an excellent response, e.g., chemotherapy naive patients, although most clinical studies report it as third-line therapy or after a failure of ¹⁷⁷Lu-PRLT. Widespread application of ²²⁵Ac-PSMA TAT is hampered by its salivary gland toxicity. The clinical studies listed above in Table 5, are described in a more detailed manner below.

In a study with the first-in-human use of ²²⁵Ac-PSMA-617 Kratochwil et al. reported complete response in two patients with mCRPC who had failed multiple lines of previous therapy. Because of challenging clinical situations and extensive pretreatment were treated with 100 kBq/kg of ²²⁵Ac-PSMA-617 at every 8 weeks as salvage therapy after the presence of a PSMA-positive tumor phenotype had been validated by ⁶⁸Ga-PSMA-11 PET/CT. The first patient was not suitable for ¹⁷⁷Lu-PSMA-617 because of widespread marrow disease and the second one progressed from ¹⁷⁷Lu-PSMA therapy presenting with diffuse abdominal and liver disease. Both patients showed a complete response on the ⁶⁸Ga-PSMA-11 PET/CT scan, and PSA declined below the measurable level. Salivary gland toxicity (xerostomia) was reported in both patients.

The second study with 14 mCRPC patients found that a treatment activity of 100 kBq/kg of body weight of ²²⁵Ac-PSMA-617 per cycle every 8 weeks was the most optimal when considering both efficacy (biochemical response) and tolerability. Severe xerostomia was the dose-limiting toxicity. The optimal dose is discussed in the dosimetry section.

Kratochwil et al. also reviewed the efficacy of ²²⁵Ac-PSMA-617 in a large cohort of 40 patients with advanced disease. All patients had mCRPC and had failed or were ineligible for conventional therapy; 70%, 85% and 60% of the patient cohort had had prior docetaxel, abiraterone and enzalutamide respectively. FIG. 3 demonstrates the therapy sequences and durations of effects in these patients on an absolute (A) and relative scale (B); the green color represents the ²²⁵Ac-PSMA-617 therapy (FIG. 3). ⁶⁸Ga-PSMA PET/CT and ^{99m}Tc-PSMA SPECT/CT imaging was used for patient selection with patients with limited disease selected for ¹⁷⁷Lu-PSMA radioligand therapy whilst those with diffuse uptake on imaging were treated with ²²⁵Ac-PSMA, those patients who demonstrated no tumor uptake on imaging were declined TAT. An activity of 100 kBq/kg ²²⁵Ac-PSMA-617 was administered for 8 weeks for a minimum 3 and up to 5 cycles.

This study demonstrated a PSA decline of more than 50% in 63% of patients, with a median duration of tumor control of 9 months. The median overall survival was more than 12 months. 38 patients out of 40 survived at least 8 weeks with 63% of these patients demonstrating a PSA response >50% and 87% demonstrating any PSA response, a median overall survival and progression free survival of >12 and 7.0 months was demonstrated. The PSA responses are shown as waterfall-plots at weeks 8, 16, 24 in FIG. 4.

This standardized treatment protocol for ²²⁵Ac-PSMA-617 is routinely applied for salvage therapy of end-stage mCRPC patients in many studies. Feuerecker et al. investigated ²²⁵Ac-PSMA-617 TAT in 26 patients who had failed a median of six lines of previous therapy for mCRPC, all had progressed after ¹⁷⁷Lu-PSMA therapy. A PSA decline of >50% was demonstrated in 65% of the patients while 88% of the patients demonstrated any PSA reduction. However, no complete response was seen in the population. The median overall survival was 7.7 months (95% CI 4.5-12.1 months). Mild irreversible xerostomia was seen in all patients with 23% of patients refusing any further treatment due to severe xerostomia, 8% of the patients had to have their treatment discontinued to prevent further deterioration of marrow toxicity which had been pre-existing. Poor prognosis could be seen in the patients who had failed previous ¹⁷⁷Lu-PSMA including the presence of liver metastases and higher ECOG status.

Similar findings were described by Yadav et al. where 28 patients with mCRPC were enrolled to receive ²²⁵Ac-PSMA-617 TAT, 54% of these patients had failed ¹⁷⁷Lu-PSMA therapy whilst 46% were ¹⁷⁷Lu-PSMA therapy naïve. A comparison of the two groups, previous ¹⁷⁷Lu-PSMA and ¹⁷⁷Lu PSMA naïve, demonstrated a PSA decline of >50% and progression rate of 26.6% and 46% vs 53.8% and 22.3% respectively. Interestingly, there was no difference in median survival and overall survival between the two groups, 10 vs. 12 months and 16 vs. 17 months, respectively.

Sathekge et al. investigated ²²⁵Ac-PSMA in 17 patients who were chemotherapy naive. ²²⁵Ac-PSMA-617 was administered in 2-monthly intervals. An initial activity of 8 MBq was administered with response assessments determined using PSA and ⁶⁸Ga-PSMA PET/CT imaging prior to subsequent cycles of ²²⁵Ac-PSMA. A “dynamic dose-reduction” was used where the subsequent activity of ²²⁵Ac-PSMA was reduced in patients who had demonstrated a response to the previous cycle, the mean administered activity was 7.4±1.5 MBq, with 3 of the 17 patients only receiving 2 cycles of therapy after having demonstrated an excellent response. A PSA decline >90% was seen in 82% of the patients at end of therapy, at median follow up of 13 months post initiation of treatment 82% of the patients were still alive with 50% of these patients in remission demonstrating undetectable serum PSA levels and the other 50% with stable disease. Grade 1-2 xerostomia was the most frequently noted side-effect with no discontinuation in therapy reported due to severe symptoms. Grade 4 nephrotoxicity was noted in a patient with only a single functional kidney who had poor renal functioning from baseline.

Sathekge et al., in the largest study population to date, enrolled 73 men with mCRPC who failed standard therapy, 14 of these patients had prior ¹⁷⁷Lu-PSMA therapy. At the end of ²²⁵Ac-PSMA-617 therapy, 70% of the patients demonstrated a ≥50% PSA decline while 82% had any decline in PSA, response shown in FIG. 5. Post therapy, ⁶⁸Ga-PSMA PET/CT images were negative in 29% of the patients. Median OS and PFS were determined to be 18 months (95% CI, 16.2-19.9 months) and 15.2 months (95% CI, 13.1-17.4 months) respectively, 13 patients had passed away while 23 patients had demonstrated disease progression. Factors found to be associated with higher OS and PFS included baseline PSA, PSA decline ≥50%, prior chemotherapy, prior radiation therapy, and Hb at baseline, while prior radioligand therapy with ¹⁷⁷Lu-PSMA was associated with a poorer PFS.

Since PSMA-617 crosses the blood-brain barrier and accumulates in cerebral metastases, a significant regression of cerebral metastases was demonstrated using ²²⁵Ac-PSMA-617. Prostate cancer patients with brain metastases have limited treatment options and poor survival, and TAT with ²²⁵Ac-PSMA-617 may have substantial therapeutic potential for these patients. Also, encouraging response to TAT in a patient with advanced mCRPC showing progression after long-term ¹⁷⁷Lu-PSMA RLT (10 cycles) has been reported (see FIG. 6). Image of the first patient exceeding 5-year complete remission after ²²⁵Ac-PSMA-TAT are shown in FIG. 7.

The patient in FIG. 6 received two cycles of ²²⁵Ac-PSMA-I&T after failure ¹⁷⁷Lu-PSMA-617 and showed encouraging response (H). The main TAT-related side effect was grade 2 xerostomia (grade 2), which was already preexisting after 10 cycles of RLT. No TAT-related grade 3/4 hematological side effects were noted.

The patient in FIG. 7 in July/September/November 2014, he received 3 cycles of mean 8.4-MBq ²²⁵Ac-PSMA-617 at PSA levels of 39.7, 7.7, and 0.32 ng/ml, respectively. This patient developed chronic xerostomia, and with some delay, creatinine increased from 1.3 in October 2015 to 3.3 mg/dl in January 2019. This could partially be related to the renal radiation exposure of PSMA therapy, but also with concomitant cardiorenal syndrome, diabetes, and arterial hypertension.

PMSA-617 has been a main theragnostic agent which has been under review in TAT in mCRPC, however PSMA-I&T has been investigated in ¹⁷⁷Lu-PSMA radioligand therapy and did not show any inferiority in the literature when compared to ¹⁷⁷Lu-PSMA-617. The first clinical data using ²²⁵Ac-PSMA-I&T showed highly comparable biochemical responses as after ²²⁵Ac-PSMA-617 TAT.

Zacherl et al. were the first to study a clinical cohort with PMSA-I&T in TAT. Fourteen patients who were either not eligible for or had failed conventional therapy were included in the study with 79% of these patients having had received prior ¹⁷⁷Lu-PSMA radioligand therapy, ¹⁸F-PSMA-1007 PET/CT was used to assess suitability for therapy. This group demonstrated a PSA decline ≥50% of 45% and any PSA decline of 73% in the subgroup of patients who had received prior ¹⁷⁷Lu-PSMA radioligand therapy which is comparable with other groups which have investigated ²²⁵Ac-PSMA therapy in patients who have failed ¹⁷⁷Lu-PSMA radioligand therapy.

Fourteen patients receiving ²²⁵Ac-PSMA-I&T were included in this retrospective analysis: Eleven of the 14 had prior second-line antiandrogen treatment with abiraterone or enzalutamide, prior chemotherapy, and prior ¹⁷⁷Lu-PSMA treatment. Patients were treated at bimonthly intervals until progression or intolerable side effects. Thirty-four cycles of ²²⁵Ac-PSMA-I&T were applied (median dose, 7.8 MBq; range, 6.0-8.5), with 1 cycle in 3 patients, 2 cycles in 7 patients, 4 cycles in 3 patients, and 5 cycles in 1 patient. No acute toxicity was observed during hospitalization. Baseline PSA was 112 ng/mL (range, 20.5-818 ng/mL). The best PSA response after TAT (a PSA decline ≥50%) was observed in 7 patients, and a PSA decline of any amount was observed in 11 patients (FIG. 8). Three patients had no PSA decline at any time. A subgroup analysis of 11 patients with prior ¹⁷⁷Lu-PSMA treatment showed any PSA decline in 8 patients and a decline of at least 50% in 5 patients; one patient is shown in FIG. 9. After TAT, grade 3 anemia was observed in 3 of the 14 patients, with 2 of them presenting with grade 2 anemia already at baseline. Grade 3 leukopenia was observed in 1 patient. Eight patients with preexisting xerostomia after ¹⁷⁷Lu-PSMA showed no worsening after TAT. Newly diagnosed grade 1 or 2 xerostomia after TAT was observed in 5 patients. One patient reported no xerostomia at all.

Radioresistance as a result of mutations in the genes responsible for DNA repair has been thought to be the reason that some patients did not demonstrate a response to ²²⁵Ac-PSMA TAT despite demonstrating tumor PSMA expression as evidenced by intense tumor uptake of tracer on PSMA PET/CT imaging. A combination of ²²⁵Ac-PSMA TAT and poly (ADP-ribose)-polymerase (PARP) inhibitors, a DNA damage-repair-targeting molecule has been suggested for these patients to overcome the radioresistance.

Kratochwil et al. identified 10 patients out of 60 who presented with a poor response to ²²⁵Ac-PSMA-617, despite sufficient tumor uptake in PSMA PET/CT. They took CT-guided biopsies with histologic validation of the nonresponding lesions in 7 of these nonresponding patients, their characteristics are shown in FIG. 10.

Specimens were analyzed by next generation sequencing (NGS) interrogating 37 DNA damage-repair-associated genes. 7 tumor samples analyzed, were found a total of 15 whole-gene deletions, deleterious or presumably deleterious mutations affecting TP53 (n=3), CHEK2 (n=2), ATM (n=2), and BRCA1, BRCA2, PALB2, MSH2, MSH6, NBN, FANCB, and PMS1 (n=1 each). The average number of deleterious or presumably deleterious mutations was 2.2 (range, 0-6) per patient. In addition, several variants of unknown significance in ATM, BRCA1, MSH2, SLX4, ERCC, and various FANC genes were detected. The summary of NGS data is shown in FIG. 11.

Patients with resistance to PSMA-TAT despite PSMA positivity frequently harbor mutations in DNA damage-repair and checkpoint genes findings encourage future studies combining PSMA-TAT and DNA damage-repair-targeting agents such as PARP inhibitors.

### d. ²²⁵Ac-PSMA TAT Toxicity Profile

Regarding salivary glands, in the clinical setting, several studies reported toxicity related to TAT with ²²⁵Ac-PSMA-617/PSMA-I&T (Table 6). FIG. 12 represents the toxicity profiles in the largest reported study with ²²⁵Ac-PSMA-617. Xerostomia is a common side effect that causes 10-25% of patients to stop TAT with ²²⁵Ac-PSMA. Xerostomia should, therefore, be prevented. Modification of the administered activity of ²²⁵Ac-PSMA-617 and the number of cycles of TAT may decrease the side effects while still achieving response. Sialendoscopy with dilatation, saline irrigation, and steroid injection (prednisolone) have been investigated in patients with some but limited success. Eleven men with metastatic castration-resistant prostate cancer underwent sialendoscopy, dilatation, saline irrigation and steroid injection of both submandibular and both parotid glands before or after every cycle of ²²⁵Ac-PSMA-617 TAT.

TABLE 6
Toxicities in 73 patients after ²²⁵Ac PSMA-617

| Toxic effect | Grade I or II | | Grade III | Grade IV |
|---|---|---|---|---|
| Dry mouth | 62 | (85) | 0 | 0 |
| Dry eyes | 4 | (5) | 0 | 0 |
| Anorexia | 23 | (32) | 0 | 0 |
| Nausea | 15 | (21) | 0 | 0 |
| Vomiting | 4 | (5) | 0 | 0 |
| Constipation | 19 | (26) | 0 | 0 |
| Fatigue | 37 | (51) | 0 | 0 |
| Weight loss | 28 | (38) | 0 | 0 |
| Dyspepsia | 3 | (4) | 0 | 0 |
| Dysgeusia | 4 | (5) | 0 | 0 |
| Anemia | 22 | (30) | 5 (7) | 0 |
| Leukopenia | 7 | (10) | 2 (3) | 0 |
| Thrombocytopenia | 6 | (8) | 1 (1) | 0 |
| Hypoalbuminemia | 14 | (19) | 0 | 0 |
| Renal Failure | 18 | (25) | 3 (4) | 2 (3) |
| Dysuria | 13 | (18) | 0 | 0 |

Data are reported as numbers of patients, with percentages of patients in parentheses.

Sialendoscopy and steroid injection were performed by a senior otolaryngologist. Quality of life (QoL) was evaluated general quality of life and specific xerostomia questionnaires, before and 3 months after the intervention. In all 11 patients, both parotid and both submandibular glands were affected by radiation sialadenitis and sialendoscopy was performed. Sialendoscopy with dilatation, saline irrigation and steroid injection had beneficial effects on salivary gland function and QoL in patients undergoing ²²⁵Ac-PSMA-617 RLT. However, even with sialadenoscopic support after multiple cycles of TAT, salivary gland function was reduced and xerostomia was present. Therefore, not only inflammation, but also the direct effect of radiation is a putative cause of dry mouth.

A case report in one patient describes the potential beneficial effects of intraparenchymal injections of botulinum toxin before ²²⁵Ac-PSMA-617 TAT. External cooling of the salivary gland using ice packs from 30 min pre-infusion through 2 h post-infusion of radiopharmaceuticals was expected to reduce PSMA radioligand uptake due to vasoconstriction. However, the relative contributions of salivary gland cooling and the reduced ²²⁵Ac-PSMA-617 activity in minimizing xerostomia severity remain unclear. Therefore, effective methods to reduce salivary toxicity are needed.

Regarding the kidneys, due to the physiological expression of PSMA in kidneys and predominantly renal excretion of 225Ac-PSMA-617, there is concern about possible radiation toxicity to the kidneys that may cause acute and long-term effects. It has been reported that the kidney function deteriorated in a patient with one functional kidney after 225Ac-PSMA-617 and that chronic kidney disease was found in two patients with mCRPC after 225Ac-PSMA-617 therapy. Until now, retention times of PSMA ligands either in kidneys or in tumor cells have not yet been evaluated systematically. If PSMA on the surface of cancer cells is not sufficiently internalized after binding of the ligand, TAT with 225Ac with multiple unstable daughters might be suboptimal and toxic. It has also been speculated that the radioactive daughters of 225Ac, but not 225Ac-PSMA-617, can accumulate in the tubular cells and irradiate the kidneys, leading to renal injury. In FIG. 13 the toxicity data has been presented in 14 patients receiving 225Ac-PSMA-I&T, in this cohort only one patient showed grade 1 nephrotoxicity.

Regarding hematologic toxicity, in FIG. 13 the hematologic toxicity in 14 patients receiving ²²⁵Ac-PSMA-I&T is mild, with single patients demonstrating grade 3 anemia and grade 3 leucopenia. The short path length of alpha particles (47-85 μm) may explain the low hematological toxicity seen in patients treated with ²²⁵Ac-PSMA, even if the marrow infiltrated by tumor cells. Baseline myelosuppression may be a contributor to increased severity of hematological toxicity. Baseline image findings of diffuse widespread marrow involvement has also been found to be predictor for hematological toxicity

Though PSMA is highly expressed prostate cancer cells, physiological expression of PSMA is seen in the lacrimal glands, salivary glands, gastrointestinal tract, and renal tubular cells. Binding to these non-malignant-tissue PSMA expressing sites is responsible for the side effects that are seen with ²²⁵Ac-PSMA therapy. Probably, therefore also xeropthalmia has been reported after ²²⁵Ac-PSMA-TAT. Safety measures that may be adopted to reduce the risk of developing nephrotoxicity include baseline screening, e.g., with ^{99m}Tc-MAG3 scintigraphy for obstructive renal pathology and correction where feasible and co-administration of normal saline with the radioligand. Patients with poor baseline renal function maybe at risk of developing severe nephrotoxicity. Most patients have demonstrated favorable nephrotoxicity to ²²⁵Ac-PSMA. However, it is known that with radionuclide therapy radiation induced renal injury may develop at a delayed stage, and thus, be missed in the reported cohorts.

Salivary gland toxicity is the most common toxicity from TAT with ²²⁵Ac-PSMA. Symptoms from xerostomia may range from mild symptoms to severe symptoms without requiring dietary changes to severe symptoms requiring nasogastric feeding or total parenteral nutrition. In a case series salivary gland toxicity was the dose limiting factor as patients refused any further treatment with ²²⁵Ac-PSMA due to intolerable xerostomia. Salivary gland toxicity is dose dependent, even irreversible severe xerostomia may develop when high cumulative activity is administered. To improve salivary gland toxicity, Sathekge et al. used a “dynamic de-escalation” where 8 MBq of ²²⁵Ac-PSMA was the initial activity. If the patient demonstrated a good response after the first cycle then the subsequent activity was reduced by 2 MBq, this process is then repeated again prior to the third cycle down to 4 MBq. This approach resulted in patients reporting only grade II xerostomia and no need for withdrawal of treatment due to salivary gland toxicity. Tandem administration of a reduced ²²⁵Ac-PMSA and full dose ¹⁷⁷Lu-PMSA has also been used as an alternative approach to reduce the severity of salivary gland toxicity without compromising PSA response.

e. Dosimetry Aspects

Dosimetry studies have demonstrated that the salivary glands receive the highest absorbed dose of the non-target organs. The mechanism of PSMA uptake in the salivary glands has not been fully understood, to date several interventions have been reviewed to improve patient quality of life, however these attempts at preventing salivary gland toxicity have been unsuccessful. These strategies have discussed earlier and in order to predict the grade of salivary gland toxicity the absorbed radiation dose should be measured.

The basis for clinical dosimetry calculations has been the relative biological effectiveness (RBE=5) found in an experimental study in a mouse model using immunohistochemical γH2AX-foci formation as an indicator for the amount of DNA double-strand breaks. The response to internal radiotherapy between α- and β-emission (²²⁵Ac/¹⁷⁷Lu) as a biological consequence of different ionization-densities along a particle-track was measured in somatostatin expressing AR42J cells which were incubated with octreotate analogs ²²⁵Ac-DOTATOC and ¹⁷⁷Lu-DOTATOC up to 48 h. The cell viability was analyzed using the common MTT assay. DNA double-strand breaks were quantified by immunofluorescence staining of γH2AX-foci and cell cycle was analyzed by flow cytometry. In vivo uptake of both radiolabeled somatostatin-analogues into subcutaneously growing AR42J tumors and the number of cells displaying γH2AX-foci were measured.

²²⁵Ac-DOTATOC resulted in ED₅₀ values of 14 kBq/ml after 48 h, whereas ¹⁷⁷Lu-DOTATOC displayed ED₅₀ values of 10 MBq/ml. The number of DNA double-strand breaks grew with increasing concentration of ²²⁵Ac-DOTATOC and similarly with ¹⁷⁷Lu-DOTATOC when applying a factor of 700-fold higher activity compared to ²²⁵Ac.

The clinical dosimetry basis and dose finding study was performed by using the isotopes ²²⁵Ac and ¹⁷⁷Lu by Kratochwil et al. A dosimetry estimate was calculated on the basis of time-activity curves derived from serially obtained ¹⁷⁷Lu-PSMA-617 scans extrapolated to the physical half-life of ²²⁵Ac, assuming instant decay of unstable daughter nuclides. Salvage therapies empirically conducted with 50 (n=4), 100 (n=4), 150 (n=2), and 200 kBq/kg (n=4) of 225Ac-PSMA-617 were evaluated retrospectively regarding toxicity and treatment response. Eight of 14 patients received further cycles in either 2- or 4-mo intervals with identical or de-escalated activities. The patient characteristics are shown in FIG. 14.

From this study, the following dosimetry estimates were observed for 1 MBq of ²²⁵Ac-PSMA-617 assuming a relative biologic effectiveness of 5: 2.3 Sv for salivary glands, 0.7 Sv for kidneys, and 0.05 Sv for red marrow that are composed of 99.4% α-, 0.5% β-, and 0.1% photon radiation, respectively. In FIG. 15, the absorbed radiation dose estimates for ²²⁵Ac-PSMA-617 are shown together with I-131-“PSMA” and ¹⁷⁷Lu-PSMA-617 doses. We see that mean dose for salivary glands is approximately 70% higher with ²²⁵Ac-PSMA-617 than with ¹⁷⁷Lu-PSMA-617, and red marrow dose 85% higher, respectively.

In clinical application, severe xerostomia became the dose-limiting toxicity if treatment activity exceeded 100 kBq/kg per cycle. At 100 kBq/kg, the duration of prostate-specific antigen decline was less than 4 months, but if therapy was repeated every 2 months patients experienced additive antitumor effects. Treatment activities of 50 kBq/kg were without toxicity but induced insufficient antitumor response in these high-tumor-burden patients. Remarkable antitumor activity by means of objective radiologic response or tumor marker decline was observed in 9 of 11 evaluable patients.

For advanced-stage patients, a treatment activity of 100 kBq/kg of ²²⁵Ac-PSMA-617 per cycle repeated every 8 weeks presents a reasonable trade-off between toxicity and biochemical response. This rationale in shown in FIG. 16.

The clinical dosimetry for ²²⁵Ac is cumbersome as discussed earlier. Even though dosimetry in clinical radionuclide therapy practice is mandatory according to EU guidelines, there are no tools available for clinical practice. One attempt has been shown in the literature. Because the ²²⁵Ac decay chain shows a noticeable gamma emission (440 keV, 25.9%; 218 keV, 11.4%). However, recommended low therapeutic activities (4-8 MBq) limit the clinical applicability of SPECT, although initial attempts for ²²⁵Ac imaging exist. Gosewisch et al. reported a mCRPC patient (65 years), whose imaging of the abdomen was performed at 24 h p. i. after therapeutic activity of 8.1 MBq ²²⁵Ac-PSMA-I&T on a SPECT/CT camera (γ-energy 440 keV; window 20%).

Final absorbed dose assessment was performed by combining the single ²²⁵Ac image with the effective half-life information determined from a previous ¹⁷⁷Lu-PSMA-I&T imaging sequence. This resulted in an absorbed dose of 0.18 and 0.17 Sv_{(RBE=5)}/MBq for the left and right kidney, respectively, compared with 0.27 and 0.24 Gy/GBq for the preceding ¹⁷⁷Lu cycle (6.2 GBq). A comparison with the pre-therapy ¹⁸F-PSMA-I&T PET/CT demonstrates that ²²⁵Ac SPECT imaging for this patient was able to locate a small lesion in the right hip. The ²²⁵Ac-absorbed dose was determined as 0.26 Sv_{(RBE=5)}/MBq, compared with 0.35 Gy/GBq for ¹⁷⁷Lu-PSMA-I&T. The imaging data is shown in FIG. 17.

All references cited herein are hereby incorporated by reference. The foregoing is offered primarily for purposes of illustration. It will be readily apparent to those skilled in the art that further drugs can be included, and that the components, additives, proportions, methods of formulation, methods of use, and other parameters described herein can be modified further or substituted in various ways without departing from the spirit and scope of the invention.

What is claimed is:

1. A pharmaceutical composition comprising:
²²⁵Actinium-PSMA-I&T in an amount that provides a radioactivity concentration of about 0.3 MBq to about 1.4 MBq per ml of the pharmaceutical composition;
ascorbic acid in an amount of about 20 mg to about 90 mg; and
ethanol in an amount of about 40 mg to about 120 mg;
wherein upon administration of the composition to a subject in need thereof, the prostate-specific antigen decline is more than about 50%.

2. The pharmaceutical composition of claim 1, wherein the radioactivity concentration is about 0.4 MBq to about 1.3 MBq per ml.

3. The pharmaceutical composition of claim 1, wherein the radioactivity concentration is about 0.5 MBq to about 1.2 MBq per ml.

4. The pharmaceutical composition of claim 1, wherein the radioactivity concentration is about 0.6 MBq to about 1.1 MBq per ml.

5. The pharmaceutical composition of claim 1, comprising from about 20 mg to about 80 mg ascorbic acid.

6. The pharmaceutical composition of claim 1, comprising from about 20 mg to about 70 mg ascorbic acid.

7. The pharmaceutical composition of claim 1, comprising from about 20 mg to about 60 mg ascorbic acid.

8. The pharmaceutical composition of claim 1, comprising from about 50 mg to about 90 mg ethanol.

9. The pharmaceutical composition of claim 1, comprising from about 50 mg to about 80 mg ethanol.

10. The pharmaceutical composition of claim 1, comprising from about 60 mg to about 80 mg ethanol.

11. A pharmaceutical composition comprising:
²²⁵Actinium-PSMA-I&T in a concentration of about 9 μg/ml to 20 μg/ml;
ascorbic acid in an amount of about 20 mg to about 90 mg; and
ethanol in an amount of about 40 mg to about 120 mg;
wherein upon administration of the composition to a subject in need thereof, the prostate-specific antigen decline is more than about 50%.

12. The pharmaceutical composition of claim 11, comprising about 11 μg/ml to about 15 μg/ml of ²²⁵Actinium-PSMA-I&T.

13. The pharmaceutical composition of claim 11, comprising from about 20 mg to about 80 mg ascorbic acid.

14. The pharmaceutical composition of claim 11, comprising from about 20 mg to about 70 mg ascorbic acid.

15. The pharmaceutical composition of claim 11, comprising from about 50 mg to about 90 mg ethanol.

16. The pharmaceutical composition of claim 11, comprising from about 60 mg to about 80 mg ethanol.

17. The pharmaceutical composition of claim 11, comprising a radioactivity concentration of about 0.3 MBq to about 1.4 MBq per ml.

18. The pharmaceutical composition of claim 11, comprising a pH of between 3 and 9.

19. The pharmaceutical composition of claim 11, comprising a pH of between 5 and 8.

20. A pharmaceutical composition comprising:
²²⁵Actinium-PSMA-I&T in an amount that provides a radioactivity concentration of about 0.5 MBq to about 1.2 MBq per ml of the pharmaceutical composition;
ascorbic acid in an amount of about 20 mg to about 70 mg; and
ethanol in an amount of about 50 mg to about 90 mg;
wherein the pharmaceutical composition comprises a pH of between 5 and 8;
wherein upon administration of the composition to a subject in need thereof, the prostate-specific antigen decline is more than about 50%.
